(19) United States
(12) Patent Application Publication
(10) Pub. No.: US 2025/0031956 A1
(43) Pub. Date: Jan. 30, 2025

(54) MEASURING APPARATUS AND MEASURING METHOD FOR PREPARING LASER TREATMENT, AND LASER TREATMENT APPARATUS

(71) Applicant: Carl Zeiss Meditec AG, Jena (DE)

(72) Inventor: Andreas BARTELS, Berlin (DE)

(21) Appl. No.: 18/696,845

(22) PCT Filed: Sep. 28, 2022
(86) PCT No.: PCT/EP2022/077055

§ 371 Date: Mar. 28, 2024

(30) Foreign Application Priority Data
Sep. 29, 2021 (DE) 10 2021 125 274.1

(51) Int. Cl. A61B 3/028; A61B 3/14; A61F 9/008; G16H 40/63

(52) U.S. Cl. CPC A61B 3/0285; A61B 3/14; A61F 9/008; G16H 40/63; A61F 2009/00878

(57) ABSTRACT

Disclosed are a measuring apparatus and a measuring method for preparing laser treatment of the eye. The measuring apparatus for preparing laser treatment has a device for determining a subjective refraction of the eye, wherein: the device for determining the subjective refraction has a housing which can be positioned in front of the eye and contains an opening; the housing has a device for providing different optical elements for the subjective visual compensation for refractive errors in the opening; and the housing is provided with a device for registration of the eye through the opening.

The invention relates to a measuring apparatus and a measuring method for preparing a laser eye treatment, a planning device for creating control data for a treatment apparatus for laser eye treatment, a treatment apparatus for laser eye treatment, and a computer program product.

In refractive laser surgery, the treatment result depends on the spatially correct application of the laser treatment on the cornea. A rotation or an offset of the laser treatment may lead to undesirable treatment results. The decisive default parameter for the laser treatment is the subjective refraction of the eye to be treated. However, the pose of head and/or eye during the measurement of the subjective refraction does not necessarily correspond to their pose during the laser treatment. In particular, a rotation of the head and/or eye cannot be ruled out. Such a relative rotation would lead to a disadvantageous treatment in the case of astigmatism since the cylinder axis during the treatment may deviate from the actual cylinder axis. Especially in the case of high cylinder values, this may lead to under corrections or optical aberrations being induced. Therefore, the preparation of laser treatments requires not only the determination of the subjective refraction but also an eye registration.

A conventional monocular subjective refraction comprises four steps performed in succession: determining the sphere, determining the cylinder axis, determining the cylinder and adjusting the sphere. In one method for determining the subjective refraction of an eye of a person, which has been known for a long time, the person gazes at a board containing letters and numbers in different sizes. Various spectacle lenses are placed systematically in front of the eyes in the process, and the person is asked whether this represents an improvement or deterioration in the visual perception. Different spectacle lenses are trialed until the person sees the optotypes on the board in focus, and hence an optimal subjective visual perception is present. This determination of the subjective refraction is performed with the aid of trial frames or what is known as a phoropter. The advantage of a phoropter is that it is possible to quickly switch between different spectacle lenses, with this being able to be implemented manually or automatically.

A phoropter typically has two housings which are arranged next to one another and positionable level with the eye. Each housing contains one or more disks or wheels which are arranged in succession and in or on which the spherical and cylindrical test lenses required to compensate refractive errors and also special media such as e.g. filters, stops, and cross cylinder lenses are arranged. The person gazes through openings in the lower part of the two housings, and the test lenses and special media are positioned in front of the openings. Switching through the test lenses can be implemented by handwheels and gearings or by motors under computer control. In the case of manually operated phoropters, the refractive powers of the test lenses located in the openings are displayed on dials which are controlled by step-by-step mechanisms; in the case of electrically controlled phoropters, the values are displayed digitally on displays or electronic visual displays. Electrically controlled phoropters, such as the digital phoropter VISUPHOR 500 by Carl Zeiss Meditec AG, offer the option of working in data sharing systems, and hence of streamlining the workflow for determining the refraction. A control device or control computer of the phoropter may be coupled to electronically operating eye refractometers, lensmeters, optotype projectors and patient/customer data systems in order to quickly adopt values of an objective refraction without errors and/or in order to be able to transmit control commands to optotype projectors and results of the determination of the subjective refraction to data systems.

A registration of the eye position, also referred to as eye registration, is required for a targeted guidance of the laser beam during a laser treatment. Eye registration is typically implemented on a piece of examination equipment, for example a wavefront aberrometer, for example WASCA by Carl Zeiss Meditec AG, which is provided with an eye registration system, for example OcuLign by Carl Zeiss Meditec AG, for the exact positioning of the wavefront data. The eye registration system records two images of the eye, one during the wavefront measurement and one just before the laser treatment. In the process, details of the iris and the limbus, i.e. the transition between cornea and sclera, are identified, and the two images are brought into correspondence.

The pose of head and/or eye may vary in the above-described measurements of the subjective refraction and eye registration. This has as a consequence that the reference image from the eye registration does not correspond, or does not correspond with sufficient precision, to the pose, in particular the rotation, of the eye during the subjective refraction.

It is an object of the present invention to specify a measuring apparatus and a measuring method for preparing a laser eye treatment, which are conducive to a correspondence between the position of the eye to be measured during the subjective refraction and the position of the eye to be measured during the eye registration.

The invention is characterized in the independent claims. The dependent claims relate to preferred developments.

An embodiment of the invention relates to a measuring apparatus for preparing a laser eye treatment, in particular a laser treatment for refractive correction of an eye, having a device for determining a subjective refraction for an eye, the device for determining the subjective refraction comprising a housing which is positionable in front of the eye and which contains an opening; the housing comprising a device for providing, in the opening, different optical elements for the subjective visual compensation of refractive errors; and the housing being provided with a device for eye registration of the eye through the opening. The eye registration device comprises an apparatus for recording a plurality of images of the eye positioned in front of the opening through the opening. An apparatus for analyzing the eye registration which is data-connected to the eye registration device is provided and designed to use the plurality of images to ascertain at least one reference image for the laser treatment.

The measuring apparatus enables both an eye registration and a determination of the subjective refraction. Hence, the eye registration and the determination of the subjective refraction can be performed with direct temporal and spatial correlation. As a result, an optimal correspondence is obtained between the position adopted by the eye to be measured during the subjective refraction and the position adopted by the eye to be measured during the eye registration. This allows the determination of the subjective refraction and the eye registration, in particular the recording and/or ascertainment of at least one eye reference image, to be performed with the same pose of the head and/or eye. The pose of the eye contains the position of the eye and/or the orientation, in particular the rotational orientation, which is also referred to as rotation, of the eye. The use of the registration information obtained thus leads to a spatially optimally oriented laser application on the cornea during the laser treatment. The measuring apparatus also allows the eye registration to be undertaken at a time of determination of the subjective refraction chosen by a user. Further, this allows the apparatus for analyzing the eye registration to ascertain at least one reference image in a pose of the eye which corresponds to a pose, in particular the rotation, of the eye during the subjective refraction.

The eye registration device can be designed to trigger a recording of the plurality of images over at least one time period. The apparatus for analyzing the eye registration can be designed to ascertain the reference image from the plurality of images or from a selection of the images by taking a time average of the images, in particular of the images of the at least one time period. The apparatus for analyzing the eye registration can be designed to ascertain the reference image from the images or a selection of the images recorded during a time period including, before or after a time which represents an optimal subjective visual compensation of refractive errors when determining the subjective refraction of the eye. The time which represents an optimal subjective visual compensation of refractive errors can be determined on the basis of measurement data from the subjective refraction, e.g. on the basis of statements regarding their visual perception given by the person whose eye is measured.

The apparatus for analyzing the eye registration can be designed to ascertain the reference image by including a tolerance range from a variation in the plurality of images. In all embodiments and modifications thereof, a variation in the plurality of images may contain and/or depict a variation in a pupil size and/or a variation of at least one iris structure. The variation in the plurality of images may result from a variation in the pupil size and/or a variation of the at least one iris structure. For example, the iris can be compressed or stretched by varying the pupil size; this may be interpreted as a rotation, also referred to as a pseudo-rotation, by an iris registration algorithm even though no rotation of the eye has occurred. Involuntary eye movements of the patient, e.g. rotations and/or tilts, which are preferably ascertained on the basis of iris structures, may also lead to an increased variation in the plurality of images. It is particularly advantageous to selectively use images from the plurality of images, in particular only use those images and/or a selection of images, for which for example the variation in the pupil size and/or of the at least one iris structure does not exceed a tolerance range. The apparatus for analyzing the eye registration may therefore be designed, in particular for ascertaining the reference image, to selectively select images from the plurality of images, in particular only select those images and/or a selection of images, for which a variation in the plurality of images does not exceed a tolerance range. The tolerance range may be predetermined.

The apparatus for analyzing the eye registration may be designed to ascertain a movement of the eye, in particular an involuntary movement of the eye, from the plurality of images. This may be implemented directly correlated in time with, in particular during, the determination of the subjective refraction. For example, the movement of the eye can be a rotation and/or a tilt of the eye. As a result, the determination of the cylinder axis in particular can be improved. For example, this is advantageous if the patient follows the settings of the optometrist too keenly during the cylinder axis determination, and the eye position during the determination of the subjective refraction deviates from the position the eye would adopt in a normal state away from the implementation of a subjective refraction.

The apparatus for analyzing the eye registration may be designed to determine a pupil size from one or more images of the eye. This may be implemented directly correlated in time with, in particular during, the determination of the subjective refraction. The pupil size influences the spherical aberration, and hence the spherical refractive value. A mean pupil size, which is taken into account when determining the subjective refraction, can be determined from several images or the plurality of images. Alternatively, it is possible to use the pupil size in the image recorded at the time when the spherical aberration was established or the cylinder axis was established.

The eye registration device can at least in part be provided together with the optical elements in the device for providing different optical elements and/or be positionable in the opening in the same manner as the optical elements. For example, the eye registration device may at least in part be provided on a disk or a wheel of a phoropter, on which optical elements required to compensate refractive errors, such as spherical and cylindrical test lenses and/or special media, e.g. filters, stops, and cross cylinder lenses, are arranged. The device for providing different optical elements may be designed in manually and/or automatically drivable fashion. For the manual provision, at least one rotary knob may be provided, by means of which a gearing for rotating at least one of the disks or at least one of the wheels of the device for providing different optical elements can be operated. For the automatic operation, a drive such as a servo motor controllable by a control device and/or a computer may be provided, by means of which the gearing for rotating at least one of the disks or at least one of the wheels of the device for providing different optical elements can be driven. The eye registration device may be provided in the housing.

In some embodiments, the eye registration device may comprise an apparatus for recording a plurality of images of the eye positioned in front of the opening through the opening. The images may capture parts of the eye, in particular the iris, parts of the sclera and/or the limbus. At least one reference image of the eye may be obtained. The apparatus for recording a plurality of images may comprise an image processing unit and/or an image display unit. The image recordings allow a registration of anatomical reference points of the eye, e.g. of iris structures and/or scleral blood vessels, correlated in space and time with the determination of the subjective refraction. The anatomical reference points of the eye obtained from the registration may be used for a greatest possible independence from the pupil size during the laser treatment. Further, it is possible to obtain a compensation of cyclorotation and displacement of the pupil center during the treatment. Moreover, a centration of the laser treatment on the basis of the limbus is enabled.

The eye registration device may comprise a mirror, at least some of which is arranged in the opening, and a camera which is arranged in or on the housing and whose optical path, in particular whose optical axis, is oriented or orientable at the mirror. In this case, the mirror may be arranged on the housing. The eye registration device may comprise a camera which is provided in or on the device for providing different optical elements and at least parts of which are positionable in the opening by means of said device for providing different optical elements. The eye registration device may comprise a camera arranged on the housing, with at least some of said camera being arranged in the opening. The camera may be arranged in the opening such that the different optical elements are provided adjacent to the camera. In these modifications, a clear view of optotypes for determining the subjective refraction can be rendered possible during the eye registration. Further, the camera can be a miniaturized camera in these modifications.

The eye registration device may comprise an apparatus for illuminating the eye positioned in front of the opening, in particular for illuminating the iris of the eye. The illumination apparatus can be designed to adapt the illuminance, e.g. by the user. The eye registration device may also comprise an apparatus for triggering the eye registration, in particular triggering the recording of the plurality of images, manually and/or automatically and/or once or multiple times. Thus, the eye registration, e.g. the recording of a reference image, may occur directly on the device for determining the subjective refraction, e.g. on a phoropter, at one or more times, defined by the user and/or automatically predetermined, of determining the subjective refraction. One or more times representing the optimal subjective visual perception may be chosen in the process. Further, the apparatus for analyzing the eye registration data-connected to the eye registration device can be designed to analyze the image quality of the recorded images of the eye, to select and/or ascertain at least one reference image for the laser treatment and/or to register anatomical reference points for the laser treatment. For example, it is possible to select the image with the best sharpness and/or illumination. The analysis of the eye registration and/or the analysis of the image quality of the recorded images of the eye and/or the selection and/or ascertainment of at least one reference image and/or the registration of anatomical reference points can be implemented automatically.

In a further embodiment, the measuring apparatus for preparing a laser eye treatment may comprise at least one element, the element being selected from a memory unit for data obtained by the measuring apparatus, a control device and an interface for transmitting data obtained by the measuring apparatus. The data obtained by the measuring apparatus may contain measurement data from the device for determining the subjective refraction, image data from the eye registration device and/or analysis data from the apparatus for analyzing the eye registration.

An embodiment relates to the use of a measuring apparatus according to any of the preceding embodiments for preparing a laser eye treatment.

An embodiment relates to a planning device for creating control data for a treatment apparatus for laser eye treatment, in particular for refractive laser eye treatment, the treatment apparatus comprising a laser device for modifying the cornea of the eye by laser beam irradiation; the planning device being designed to determine control data for the laser device of the treatment apparatus; wherein the planning device comprises an interface for receiving data obtained by the measuring apparatus according to any of the embodiments for preparing a laser eye treatment, and wherein the planning device is designed to use the data from the measuring apparatus received via the interface to create the control data for the treatment apparatus. The planning device may be data-connected via the interface for receiving data to the measuring apparatus according to any of the embodiments for preparing a laser eye treatment. The planning device may comprise an interface for transmitting created control data. The treatment apparatus may be a treatment apparatus according to embodiments.

A further embodiment relates to a method for creating control data for a treatment apparatus for laser eye treatment, especially using the planning device of the preceding embodiment. In this case, control data for the laser device are calculated using data obtained by the measuring apparatus according to any of the embodiments for preparing a laser eye treatment. An embodiment specifies a computer program product having one or more program codes which, when executed on a computer, carry out the method for creating the control data according to the preceding embodiment. The planning device of embodiments may be designed as a computer.

Another embodiment specifies a treatment apparatus for laser eye treatment, in particular for refractive laser eye treatment, having a laser device for modifying the cornea of the eye by laser beam irradiation; wherein the treatment apparatus comprises at least one element selected from a measuring apparatus according to any of the embodiments for preparing a laser eye treatment and a planning device according to any of the embodiments for creating control data for a treatment apparatus for laser treatment. The laser device, the measuring apparatus, the planning device and/or a central control device of the treatment apparatus may be data-connected to one another in wired or wireless fashion, in particular by way of interfaces. The treatment apparatus may comprise one or more interfaces for receiving data, e.g. control data from the planning device of embodiments and/or data obtained by the measuring apparatus of embodiments.

An additional embodiment relates to a method for laser eye treatment, in particular for refractive laser treatment, in particular using a treatment apparatus according to the preceding embodiments, wherein the cornea of an eye is modified by laser beam irradiation using data obtained by the measuring apparatus according to any of the embodiments for preparing a laser eye treatment. A further embodiment relates to a computer program product comprising at least one program code causing the treatment apparatus according to any of the preceding embodiments to carry out the steps of the laser treatment method according to the preceding embodiment, especially if the program code is loaded into a memory unit of the treatment apparatus.

A further embodiment relates to a measuring method for preparing a laser eye treatment, in particular a laser treatment for refractive correction of an eye, wherein an eye registration of an eye is performed together with a provision of different optical elements for the subjective visual compensation of refractive errors. In particular, the eye registration can be implemented with direct temporal and spatial correlation with the provision of different optical elements for the subjective visual compensation of refractive errors, e.g. during or immediately after the determination of the subjective refraction.

The measuring method is carried out using a measuring apparatus according to any of the previous embodiments for preparing a laser eye treatment. The eye registration of the eye is implemented through the opening. Hence, the eye registration can be implemented in the same position of the eye as during the determination of the subjective refraction.

The eye registration is further implemented by recording a plurality of images of the eye positioned in front of the opening. At least one reference image for the laser treatment is ascertained on the basis of the plurality of images. This step can be implemented during the eye registration and/or during an analysis of the eye registration.

A recording of the plurality of images can be triggered over at least one time period. The reference image can be ascertained from the plurality of images or from a selection of the images by taking a time average of the images, in particular of the images of the at least one time period. The reference image can be ascertained from the images or a selection of the images recorded during a time period including, before or after a time which, especially when determining the subjective refraction of the eye, represents an optimal subjective visual compensation of refractive errors.

The reference image can be ascertained by including a tolerance range from a variation in the plurality of images. A variation in the plurality of images may contain and/or depict a variation in a pupil size and/or a variation of at least one iris structure. The variation in the plurality of images may result from a variation in the pupil size and/or a variation of the at least one iris structure. In the measuring method, in particular for ascertaining the reference image, it is possible to selectively select images from the plurality of images, in particular only select those images and/or a selection of images, for which a variation in the plurality of images does not exceed a tolerance range. The tolerance range may be predetermined. These steps can be implemented during the eye registration and/or during the analysis of the eye registration.

A movement of the eye, in particular an involuntary movement of the eye, can be ascertained from the plurality of images. This may be implemented directly correlated in time with, in particular during, the determination of the subjective refraction. For example, the movement of the eye can be a rotation and/or a tilt of the eye. As a result, the determination of the cylinder axis in particular can be improved. These steps can be implemented during the eye registration and/or during the analysis of the eye registration.

Further, a pupil size can be determined from one or more images of the eye. This may be implemented directly correlated in time with, in particular during, the determination of the subjective refraction. A mean pupil size, which is taken into account when determining the subjective refraction, can be determined from several images or the plurality of images. Alternatively, it is possible to use the pupil size in the image recorded at the time when the spherical aberration was established or the cylinder axis was established. These steps can be implemented during the eye registration and/or during the analysis of the eye registration.

The eye registration can be implemented by means of a mirror, at least some of which is arranged in the opening, and a camera which is arranged in or on the housing, wherein the optical path, in particular the optical axis, of the camera is oriented at the mirror. The eye registration can be implemented by means of a camera which is provided in or on the device for providing different optical elements and at least parts of which are positioned in the opening by means of said device for providing different optical elements. The eye registration may be implemented by means of a camera arranged on the housing, with at least some of said camera being arranged in the opening. The eye, in particular the iris of the eye, positioned in front of the opening can be illuminated. The eye registration, in particular the recording of the images, can also be triggered manually and/or automatically and/or once or multiple times.

Moreover, the image quality of the recorded images of the eye can be analyzed. Further, at least one reference image can be selected or ascertained for the laser treatment and/or anatomical reference points can be registered for the laser treatment. Each of these steps can be implemented automatically. These steps can be implemented during the eye registration and/or during the analysis of the eye registration.

A further embodiment relates to a computer program product comprising at least one program code causing the measuring apparatus according to any of the preceding embodiments and modifications and/or the treatment apparatus according to any of the preceding embodiments and modifications to carry out the steps of the measuring method according to any of the preceding embodiments and modifications, especially if the program code is loaded into a memory unit of the measuring apparatus and/or treatment apparatus.

The laser eye treatment can be a refractive laser eye treatment in all embodiments.

With the measuring method for preparing a laser eye treatment, it is possible to realize the same advantages, modes of operation and functions as with the embodiments and modifications of the measuring apparatus for preparing a laser eye treatment, especially with equivalent and/or analogous features. The same applies to the planning device of embodiments for creating control data for a treatment apparatus for laser eye treatment and to the treatment apparatus of embodiments for laser eye treatment, and to the method of embodiments for creating control data and the method of embodiments for laser eye treatment.

It goes without saying that the features mentioned above and the features yet to be explained hereinafter can be used not only in the specified combinations but also in other combinations or on their own, without departing from the scope of the present invention.

The invention will be explained in more detail below on the basis of exemplary embodiments, with reference being made to the appended drawings, which likewise disclose features essential to the invention. These exemplary embodiments are only illustrative and should not be construed as restrictive. For example, a description of an exemplary embodiment having a multiplicity of elements or components should not be construed as meaning that all of these elements or components are necessary for implementation. Rather, other exemplary embodiments may also contain alternative elements and components, fewer elements or components, or additional elements or components. Elements or components of different exemplary embodiments can be combined with one another, unless indicated otherwise. Modifications and variations that are described for one of the exemplary embodiments can also be applicable to other exemplary embodiments. In order to avoid repetition, elements that are the same or correspond to one another in different figures are denoted by the same reference signs and are not explained repeatedly. In the figures:

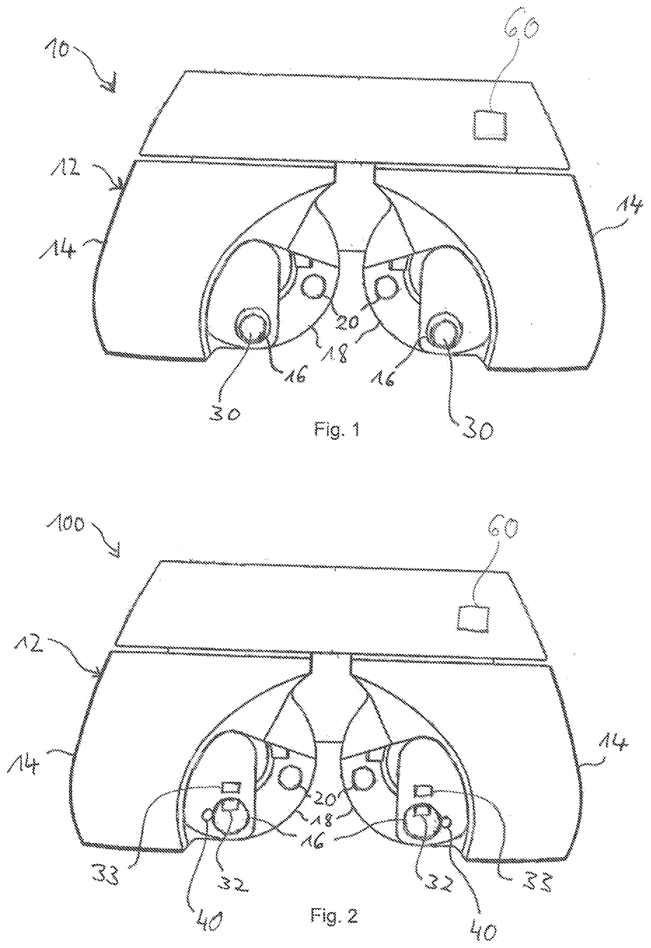

FIG. 1 schematically shows a first example of a measuring apparatus for preparing a laser eye treatment;

FIG. 2 schematically shows a second example of a measuring apparatus for preparing a laser eye treatment;

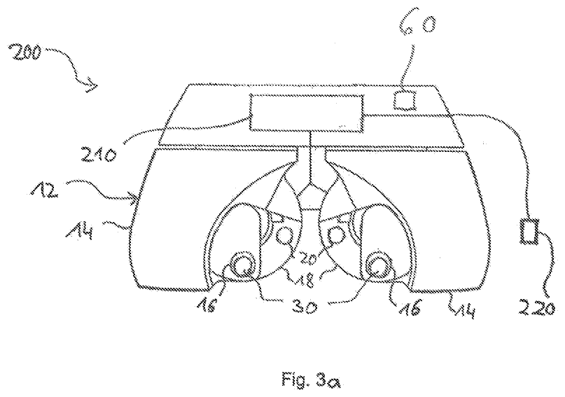

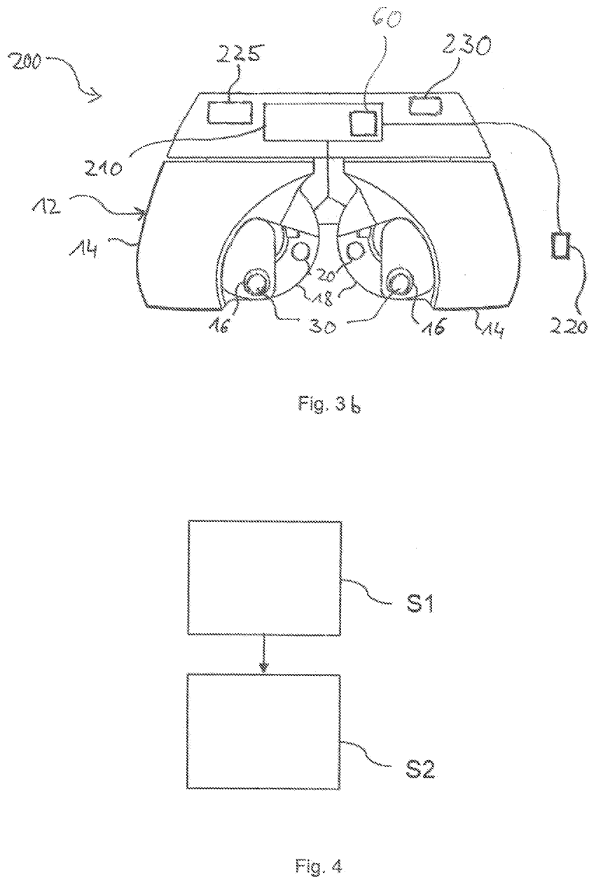

FIG. 3a schematically shows a third example of a measuring apparatus for preparing a laser eye treatment;

FIG. 3b schematically shows a modification of the third example;

FIG. 4 schematically shows an example of a measuring method for preparing a laser eye treatment;

FIG. 5 schematically shows an example of a planning device for creating control data for a treatment apparatus for laser eye treatment; and

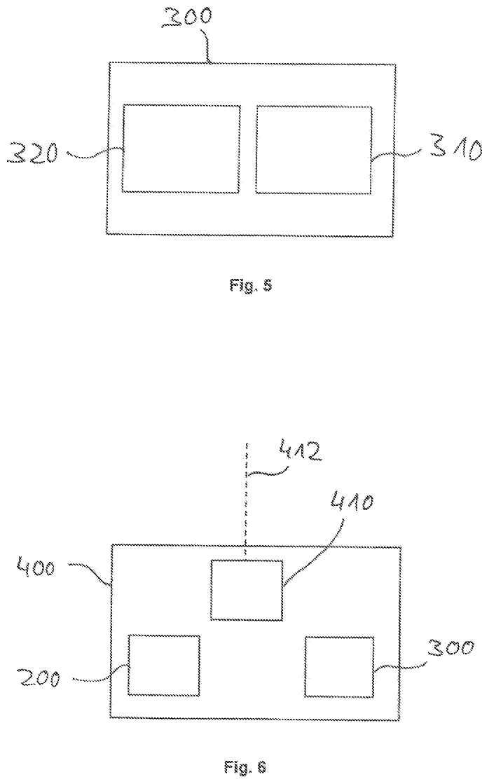

FIG. 6 schematically shows an example of a treatment apparatus for laser eye treatment.

In the present case, the phrase “an eye registration of an eye is performed together with a provision of different optical elements for the subjective visual compensation of refractive errors” should be understood as meaning “an eye registration is performed with a temporal and spatial correlation with a provision of different optical elements for the subjective visual compensation of refractive errors”, e.g. during or immediately after an automated provision of different optical elements for the subjective visual compensation of refractive errors. Further, the phrase “the device for determining the subjective refraction comprises a housing which is positionable in front of the eye and which contains an opening” also comprises a device having two such housings which are positionable next to one another, in each case in front of an eye of a person. The terms “measurement” and “measure” and terminological modifications thereof presently contain a collection of data, which contain measurement data and/or image data, e.g. data from anatomical reference points of the eye based on a reference image, and/or analysis data. The data contain measurement data from the subjective refraction, e.g. statements regarding their visual perception by a person whose eye is measured, image data from the eye registration and/or analysis data from the apparatus for analyzing the eye registration. The term “opening” can be understood to mean a passage opening in the housing or a window in the housing, by means of which it is possible to gaze through the housing with the eye to be measured. The terms “during a subjective refraction”, “during a measurement of the subjective refraction” or “during a determination of the subjective refraction” and modifications thereof can be understood to be synonymous.

As a first example, FIG. 1 shows a measuring apparatus 10 for preparing a laser eye treatment, in particular a laser treatment for refractive correction of an eye. The measuring apparatus 10 comprises a device 12 for determining a subjective refraction, having two housings 14 which are arranged next to one another and are positionable level with the eye at an intraocular distance from one another. To position the housings 14 level with the eye, the measuring apparatus is provided with a height-adjustable frame (not shown here). For positioning at an intraocular distance from one another, the housings 14 are pivotable relative to one another. The housings 14 each comprise an opening 16, in front of which the eye to be measured can be held. In the present case, the measuring apparatus 10 is a phoropter. In other examples, the device 12 for determining the subjective refraction contains only one housing 14, which is positionable at eye level with an eye to be measured.

Each housing 14 contains a device with which different optical elements 20 for the subjective visual compensation of refractive errors can be provided in the respective opening 16. In the present example, the device for providing different optical elements is in the form of a disk 18 with a circular circumference, which can be rotated manually within the housing 14. A plurality of optical elements 20 are provided in the disk 18 in a manner distributed over a circle and are arranged such that they are positionable in the opening 16 by rotating the disk 18. The optical elements 20 are spherical and/or cylindrical test lenses required to compensate refractive errors, e.g. lens elements, and/or special media, e.g. filters, stops, and cross cylinder lenses.

A device for eye registration of an eye is provided in each housing 14. In the present case, the eye registration device is in the form of a miniaturized camera 30. The object lens and the sensor of the miniaturized camera 30 are designed in such a way in the present example that the entire eye of the patient is captured, and imaging is in focus in the case of desired positioning of the eye at a vertex distance (HSA) from the phoropter. The focus of the object lens is approximately at a distance of 10 to 20 mm, for a typical vertex distance of 12 to 16 mm. From a technical point of view, this is realized in the present example by a fixed focus with a deep depth of field or an autofocus. In the present case, a 2 MP (megapixel) sensor is used to provide the resolution of the camera. As a result, the resolution of the camera enables reliable identification of the required iris structures and scleral vessels. The miniaturized camera 30 is provided on the disk 18, i.e. on the device for providing different optical elements, on the circle between the optical elements 20. The miniaturized camera 30 is positionable in the opening 16 by means of the disk 18. The miniaturized camera 30 serves to record, through the opening 16, a plurality of images of the eye positioned in front of the opening 16. To this end, the miniaturized camera 30 on the disk 18 is oriented such that its optical path runs substantially perpendicular to a plane of the opening 16. Further an apparatus 60 for analyzing the eye registration which is data-connected to the eye registration device 30 in wired or wireless fashion is provided and designed to use the images to ascertain at least one reference image for the laser treatment.

The apparatus 60 for analyzing the eye registration can be designed to ascertain the reference image from the images or from a selection of the images by taking a time average of the images, in particular of the images of the at least one time period. The apparatus for analyzing the eye registration can also be designed to ascertain the reference image from a selection of the images recorded during a time period including, before or after a time which represents an optimal subjective visual compensation of refractive errors when determining the subjective refraction of the eye. The apparatus 60 for analyzing the eye registration can be designed to ascertain the reference image by including a tolerance range from a variation in the images or a selection thereof. In this case, the apparatus 60 for analyzing the eye registration may be designed to selectively select images from the plurality of images, in particular only those images and/or a selection of images, for which a variation of the plurality of images, in particular a variation in the pupil size and/or of at least one iris structure, does not exceed a tolerance range. The apparatus 60 for analyzing the eye registration may also be designed to ascertain a movement of the eye, in particular an involuntary movement of the eye, from the plurality of images when determining a subjective refraction. The apparatus 60 for analyzing the eye registration may moreover be designed to determine a pupil size from one or more images of the eye when determining the subjective refraction.

Measurements for preparing a laser eye treatment are undertaken using the measuring apparatus 10. A subjective refraction of one or both eyes of a person is determined, i.e. measured, in the process, and an eye registration is performed. Manual adjustment of the disk 18 leads to different optical elements 20 being positioned in the relevant opening 16 for an eye of the person. The subjective refraction of the eye is determined using statements by the person regarding their resultant visual perception. Determining the subjective refraction comprises the steps of determining the sphere, determining the cylinder axis, determining the cylinder and adjusting the sphere. The eye registration is undertaken at one or more times during the determination of the subjective refraction which are established by a user, e.g. the operator of the measuring apparatus 10 and/or the person whose eye is measured. As a result, the eye registration is implemented with the same pose of head and/or eye as the subjective refraction. For example, a time at which the optimal visual perception of the person is established within the scope of the subjective refraction is chosen for the eye registration. In the present example, the eye registration is performed immediately following the completion of the subjective refraction. The eye registration is implemented by manually adjusting the disk 18 and positioning the miniaturized camera 30 in the opening 16 and using the miniaturized camera 30 to record a plurality of images of the eye positioned in front of the opening. The recordings of the camera 30 are triggered by the user, e.g. by way of a remote control. Further, the camera 30 may be assigned an image processing unit and an image display unit (neither of which are shown) in a manner for exchanging data, said units being data-connected to or integrated in the apparatus 60 for analyzing the eye registration. At least one reference image is ascertained using the apparatus 60 for analyzing the eye registration.

The measuring apparatus 10 optionally comprises a light source (not shown in FIG. 1) as an apparatus for illuminating the eye positioned in front of the opening. The latter is arranged next to the opening 16 on the housing 14 and enables the illumination of the eye to be measured, e.g. of the iris, in the first example. The illuminance may be adjustable at the light source. The light source enables a standardized illumination of the eye at the time of recording. Infrared light-emitting diodes which, with diffuse light, enable an illumination of the eye that is as uniform as possible and adapted to the sensitivity of the camera can be used as the light source.

FIG. 2 shows, as a second example, a measuring apparatus 100 for preparing a laser eye treatment. Instead of the miniaturized camera 30, a mirror 32 that is permanently arranged in the opening 16 and a camera 33 that is permanently arranged on the outer side of the housing 14 are provided, on each housing 14 of the measuring apparatus 100, as the device for the eye registration of an eye. The camera 33 may also be provided within the housing 14. The optical path of the camera 33 is directed at the mirror 32 while the mirror 32 is oriented at the position of the eye to be measured in front of the housing 14. In the present example, the opening 16 has a size and/or shape allowing the optical elements 20 to be positioned next to the mirror 32 within the opening 16. As a result, the person is afforded a clear view of optotypes for determining the subjective refraction during the eye registration. A light source 40 designed like the optional light source of the first example is provided next to the opening 16. The camera 33 is designed like the miniaturized camera 30 of the first example. Further, the apparatus 60 for analyzing the eye registration is provided, data-connected to the eye registration device 32, 33 and designed to ascertain at least one reference image for the laser treatment on the basis of the images.

The camera 33 is arranged on the housing in the opening 16 instead of the mirror 32 in a modification of the second example. Consequently, no mirror is present in this modification. The camera 33 is provided in the opening 16 such that the different optical elements 20 can be provided adjacent to the camera 33.

In a further modification of the second example, the camera 33 is sensitive in the infrared range, e.g. in a wavelength range from 800 to 1000 nm, and comprises a filter which only transmits infrared light and filters out visible light. Further, the light source 40 next to the opening 16 is a light source emitting infrared light in the wavelength range of the camera 33, for example infrared light-emitting diodes. As a result of the infrared illumination, the ambient light striking the eye through the opening 16 has a lesser influence on the image quality, and the person whose eye is measured does not perceive the infrared light as bothersome. In an additional revision of this modification, the mirror 32 is transparent to visible light and only reflects infrared light. Together with the infrared camera 33 and the infrared illumination, this is conducive to a clear view of the patient through the opening 16 when recording a camera image at the same time.

A subjective refraction is determined and an eye registration is performed using the measuring apparatus 100. Manual adjustment of the disk 18 leads to different optical elements 20 being provided in the opening 16 for each eye to be measured. The eye registration is carried out with the camera 33 and via the mirror 32 at one or more desired times during the determination of the subjective refraction. In the process, a plurality of images are recorded of the eye which is positioned in front of the opening and illuminated by the light source 40. A plurality of images are recorded automatically when the camera 33 is triggered. Taking account of the mirrored recording, one or more images of the eye are provided using an image processing unit and/or an image display unit (neither of which are shown) which are assigned to the camera 33 in a manner for exchanging data and are data-connected to or integrated in the apparatus 60 for analyzing the eye registration. At least one reference image is ascertained using the apparatus 60 for analyzing the eye registration.

FIG. 3a shows, as a third example, a measuring apparatus 200 for preparing a laser eye treatment. The measuring apparatus 200 is designed like the measuring apparatus 10, but additionally contains a control device 210 which is data-connected to a controllable drive (not shown), e.g. a servo motor, of the disk 18 and which controls said drive. Further, an apparatus 220 for manually triggering the eye registration device is provided and data-connected to the control device 210. In the present example, the apparatus 220 is embodied as a pushbutton that is data-connected to the control device 210. The control device 210 is also data-connected to the camera 30, to the apparatus 60 for analyzing the eye registration and to an image processing unit and an image display unit (neither of which units are shown) which are assigned to the camera 30. The image processing unit may be contained in the control device 210. The data-connections are configured in wired or wireless fashion.

A determination of a subjective refraction and an eye registration are performed using the measuring apparatus 200. Adjustment of the disk 18 by means of the control device 210 leads to different optical elements 20 being provided in the opening 16 for each eye to be measured. The camera 30 is used to undertake the eye registration at one or more desired times during the provision of the optical elements 20, wherein one or more images of the eye positioned in front of the opening are recorded using the camera 30. In this case, the eye registration is initiated manually using the pushbutton 220, for example by the person whose eye is measured. This causes the camera 30 to be positioned in the opening 16 and be triggered. The recorded images of the eye are stored in a memory unit (not shown) of the measuring apparatus 200 and are displayed on the image display unit. The memory unit may be integrated in the control device 210. The operator of the measuring apparatus 200 can select the image with the best sharpness and/or illumination. The operator or the person whose eye is measured can repeat the image recording any desired number of times, until a suitable image is available. According to a further example, a plurality of images are automatically recorded by means of the control device 210 when the camera 30 is triggered. At least one reference image is ascertained using the apparatus 60 for analyzing the eye registration.

FIG. 4 shows an example of a measuring method for preparing a laser eye treatment using a modification of the measuring apparatus 200. The control device 210 of the measuring apparatus 200 is used in a step S1 to automatically provide different optical elements 20 for the subjective visual compensation of refractive errors and in a step S2 to automatically initiate the eye registration at predetermined times during the determination of the subjective refraction. In this case, the disk 18 is automatically repositioned in step S1 by means of the control device 210 and the different optical elements 20 are presented in the opening 16. The statements by the person regarding their resultant visual perception can be input and stored by means of an input apparatus (not shown) of the control device 210. In step S2, the camera 30 is triggered by means of the control device 210 at the predetermined times and at least one reference image is ascertained using the apparatus 60 for analyzing the eye registration.

According to a modification of the third example illustrated in FIG. 3b, the control device 210 of the measuring apparatus 200 contains the apparatus 60 for analyzing the eye registration, which is data-connected to the eye registration device and which is designed to ascertain at least one reference image for the laser treatment on the basis of the plurality of images. Alternatively, the control device 210 is designed as the apparatus 60 for analyzing the eye registration. In particular, the apparatus 60 is designed to analyze the image quality of the recorded images of the eye and/or to select at least one reference image for the laser treatment. During operation, the ascertainment of the at least one reference image for the laser treatment, the analysis of the image quality of the recorded images of the eye and/or the selection of a reference image are implemented automatically by means of the control device 210 and/or the apparatus 60. These and other steps of the analysis of the eye registration are integrated in step S2 of FIG. 4. In the modification of the third example, the measuring apparatus 200 comprises a memory unit 225 for data obtained by the measuring apparatus and for statements given by the person regarding their visual perception, and an interface 230 for transmitting data obtained by the measuring apparatus.

The measuring apparatus 100 of the second example can be modified in the same way as the measuring apparatus 200 of the third example. For example, the measuring apparatus 100 may be equipped with the control device 210 and/or the apparatus 220 for manually triggering the eye registration and may be operable in a corresponding manner.

FIG. 5 schematically shows an example of a planning device 300 for creating control data for a treatment apparatus for laser eye treatment, in particular for refractive laser eye treatment. An example of a treatment apparatus 400 for laser treatment, schematically illustrated in FIG. 6, comprises a laser device 410 for modifying the cornea of the eye by laser beam 412 irradiation.

The planning device 300 is designed to determine control data for the laser device 410 of the treatment apparatus 400. As shown in FIG. 5, the planning device 300 comprises an interface 310 for receiving data obtained for example by the measuring apparatus 200 of the third example. The data can be input at the interface 310, and/or the interface 310 may be data-connected to the measuring apparatus 200. These data are used by the planning device 300 to create the control data. The planning device 300 also comprises an interface 320 for transmitting control data created, by means of which the control data can be transmitted to the treatment apparatus 400 for laser treatment, for example.

In the example of FIG. 6, the treatment apparatus 400 for laser eye treatment contains the measuring apparatus 200 of the third example and the planning device 300 for creating control data. The measuring apparatus 200 and the planning device 300 and also the laser device 410 and the planning device 300 are data-connected to one another in wireless or wired fashion, for example via a central control device (not shown) of the treatment apparatus 400 and via the interfaces 230, 310, 320.

1.-14. (canceled)

15. A measuring apparatus that facilitates preparing a laser eye treatment, comprising:
a device that facilitates determining a subjective refraction of an eye;
the device that facilitates determining the subjective refraction comprising a housing which is positionable in front of the eye and which contains an opening; the housing comprising a device that provides, in the opening, different optical elements for subjective visual compensation of refractive errors;
wherein
the housing includes an eye registration device that registers the eye through the opening;
the eye registration device comprising a recording apparatus that records a plurality of images of the eye, wherein the recording apparatus is positioned in front of the opening and captures the images of the eye through the opening; and
a computer analysing apparatus that analyses the eye registration and which is data-connected to the eye registration device and is provided and configured to use the plurality of images to ascertain at least one reference image for the laser eye treatment.

16. The measuring apparatus as claimed in claim 15, wherein the eye registration device is configured to trigger a recording of the plurality of images over at least one time period.

17. The measuring apparatus as claimed in claim 16,
wherein the analysing apparatus that analyzes the eye registration is configured to ascertain the reference image from the plurality of images or from a selection of the images by taking a time average of the images; or
wherein the analyzing apparatus that analyzes the eye registration is configured to ascertain the reference image from the images or a selection of the images recorded during a time period including, before or after a time which represents an optimal subjective visual compensation of refractive errors when determining the subjective refraction of the eye; or
wherein the apparatus for analyzing the eye registration is configured to ascertain the reference image by including a tolerance range from a variation in the plurality of images; or
a combination of the foregoing.

18. The measuring apparatus as claimed in claim 17, wherein the selection of the images is from at least one time period.

19. The measuring apparatus as claimed in claim 15,
wherein the eye registration device comprises a mirror, at least a portion of which is arranged in the opening, and a camera which is arranged in or on the housing and whose optical path is oriented at or orientable at the mirror; or
wherein the eye registration device comprises a camera which is provided in or on the device that provides different optical elements and which is positionable in the opening by operation of said device that provides different optical elements; or
wherein the eye registration device comprises a camera arranged on the housing in the opening; or a combination of the foregoing.

20. The measuring apparatus as claimed in claim 15,
wherein the eye registration device comprises an apparatus that illuminates the eye positioned in front of the opening, including illuminating the iris of the eye; or
wherein the eye registration device comprises an apparatus that triggers the eye registration, in particular triggering the recording of the plurality of images, manually, automatically and once or multiple times; or
wherein the apparatus for analyzing the eye registration data-connected to the eye registration device is configured to analyze the image quality of the recorded images of the eye, to select at least one reference image for the laser treatment, to register anatomical reference points for the laser treatment, or
a combination of the foregoing.

21. The measuring apparatus as claimed in claim 15,
wherein the measuring apparatus comprises at least one element selected from a group consisting of a memory unit that stores data obtained by the measuring apparatus, a control device and an interface that transmits data obtained by the measuring apparatus.

22. A computerized planning device that creates control data for a treatment apparatus that performs laser eye treatment, including refractive laser eye treatment,
the treatment apparatus comprising a laser device that modifies a cornea of the eye by application of laser beam irradiation;
the planning device being configured to determine control data for the laser device of the treatment apparatus;
wherein
the planning device comprises an interface for receiving data obtained by the measuring apparatus as claimed in claim 15 for preparing a laser eye treatment, and
the planning device is configured to use the data from the measuring apparatus received via the interface to create the control data for the treatment apparatus.

23. A treatment apparatus that performs laser eye treatment, including refractive laser eye treatment, comprising:
a laser device that modifies the cornea of the eye by laser irradiation;
wherein
the treatment apparatus comprises a planning device as claimed in claim 22 for creating control data for a treatment apparatus for laser treatment.

24. A measuring method for preparing a laser eye treatment, comprising:
performing an eye registration of an eye together with a provision of different optical elements for the subjective visual compensation of refractive errors;
performing the measuring method using a measuring apparatus as claimed in claim 15 to prepare a laser eye treatment;
implementing the eye registration of the eye through the opening and recording a plurality of images of the eye positioned in front of the opening through the opening; and
ascertaining at least one reference image for the laser treatment on the basis of the plurality of images.

25. The measuring method as claimed in claim 24, further comprising triggering a recording of the plurality of images over at least one time period.

26. The measuring method as claimed in claim 15, further comprising:
ascertaining the reference image from the plurality of images or from a selection of the plurality of images by taking a time average of the images of the images of the at least one first time period; or
ascertaining the reference image from the images or a selection of the images recorded during a second time period including, before or after a time which represents an optimal subjective visual compensation of refractive errors; or
ascertaining the reference image by including a tolerance range from a variation in the plurality of images; or
a combination of the foregoing.

27. The measuring method as claimed in claim 24, further comprising:
implementing the eye registration by application of a mirror, at least a portion of which is arranged in the opening, and a camera which is arranged in or on the housing and an optical path of which is oriented at the mirror; or
implementing the eye registration using a camera arranged in or on the device that provides different optical elements and which is positioned in the opening by operation of said device for providing different optical elements; or
implementing the eye registration using a camera arranged on the housing, with at least a portion of the camera being arranged in the opening; or
illuminating the eye, including the iris of the eye, positioned in front of the opening; or
triggering the eye registration, including the recording of the images, manually or automatically or once or multiple times; or
a combination of the foregoing.

28. The measuring method as claimed in claim 24, further comprising:
analyzing the image quality of the recorded images of the eye; or
selecting at least one reference image for the laser treatment; or
registering anatomical reference points for the laser treatment; or
a combination of the foregoing.
